SEQUENCE LISTING (9 sequences)

SEQ ID NO: 1 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 292 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 3 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 4 - protein, 311 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 5 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 6 - protein, 274 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 7 - protein, 287 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 8 - protein, 293 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 9 - protein, 318 aa, synthetic construct
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNRGGGGSEQKLISEEDL

(19) United States
(12) Patent Application Publication
(10) Pub. No.: US 2025/0281420 A1
(43) Pub. Date: Sep. 11, 2025

(54) MACROPHAGE IMMUNOTHERAPY

(71) Applicants: University of Massachusetts, Boston, MA (US); Volvox Sciences LLC, Middletown, DE (US)

(72) Inventors: Ashish Kulkarni, Arlington, MA (US); Samia Shamim, London (GB)

(21) Appl. No.: 18/861,373

(22) PCT Filed: May 5, 2023
(86) PCT No.: PCT/US2023/066703

§ 371 Date: Oct. 29, 2024

Related U.S. Application Data
(60) Provisional application No. 63/364,221, filed on May 5, 2022.

(51) Int. Cl. A61K 9/51; A61K 38/17; A61K 39/395; A61P 35/00

(52) U.S. Cl. CPC A61K 9/5123; A61K 38/1774; A61K 39/39558; A61P 35/00

(57) ABSTRACT

The present disclosure provides an engineered amphiphilic composition (for example, a lipid nanoparticle) comprising multiple CD47 proteins and at least one amphiphile. The disclosure further provides methods for enhancing the phagocytic function of macrophages by contacting the macrophages with macrophages with the engineered amphiphilic composition. The disclosure further provides methods for treating cancer by administering the engineered amphiphilic composition to a patient in need thereof.

### PRIORITY

This application claims the benefit of priority to U.S. Provisional Patent Application Ser. No. 63/364,221, filed May 5, 2022, the content of which is incorporated herein by reference in its entirety.

### REFERENCE TO A SEQUENCE LISTING

This application incorporates by reference the Sequence Listing submitted in XML as file Sequence_Listing_3724-080PCT, created on May 5, 2023 and containing 11,443 bytes.

### FIELD

The field of the present disclosure relates to compositions and methods for treating cancers. In particular, contemplated are engineered amphiphilic compositions, such as lipid nanoparticles (LNPs), comprising multiple CD47 proteins, and the use of such compositions for treating cancer.

### BACKGROUND

Macrophages are cells of the body that exist in either a pro inflammatory M1 phenotype capable of mounting an attack against incoming pathogens or can exist in an anti-inflammatory M2 phenotype that maintains homeostasis in tissues. However, in many solid tumors, the tumor associated macrophages (TAMs) exist in an M2 phenotype, which is pro tumorigenic in nature and supports the growth and metastasis of tumors.

### SUMMARY

Provided herein is an engineered amphiphilic therapeutic composition, wherein the engineered amphiphilic composition comprises multiple CD47 proteins (mCD47/multivalent) and at least one amphiphile, wherein the engineered amphiphilic composition binds SIRP-alpha protein. In one aspect, the at least one amphiphile comprises one or more of 1,2-Distearoyl-sn-glycero-3-phosphoethanolamine (DSPE), cholesterol, sitosterol, phosphatidylcholine (PC) or other lipids that are conjugable. In another aspect, the amphiphile is pegylated. In one aspect, the engineered amphiphilic composition further comprises a carrier. In another aspect, the engineered amphiphilic composition further comprises one or more additional anticancer agents.

In embodiments, the CD47 protein is conjugated to the amphiphile. In embodiments, the CD47 protein comprises the amino acid sequence of one or more of SEQ ID NOs: 1-8.

In one aspect, the engineered amphiphilic composition comprises one or more phospholipids, one or more pegylated phospholipids and cholesterol. In embodiments, the pegylated phospholipid(s), cholesterol, and phospholipid(s) may be present in molar ratios of about 1:1:8, about 1:3:6, or about 1:4:5. In embodiments, the pegylated phospholipid(s) comprise about 5 to about 30 mol % of the lipid component; the cholesterol comprises about 5 to about 40 mol % of the lipid component; and the phospholipid(s) comprise about 40 to about 80 mol % of the lipid component.

In one embodiment, the engineered amphiphilic composition comprises one or more of L-α-phosphatidylcholine (PC), Cholesterol, and 1,2-Distearoyl-sn-Glycero-3-Phosphoethanolamine-N-[Carboxy (Polyethylene Glycol)2000] (DSPE-PEG-Carboxylic Acid) or DSPE-PEG-maleimide. In one embodiment, the engineered amphiphilic composition comprises L-α-phosphatidylcholine (PC), Cholesterol, and 1,2-Distearoyl-sn-Glycero-3-Phosphoethanolamine-N-(Carboxy (Polyethylene Glycol)2000) (DSPE-PEG-Carboxylic Acid). In one embodiment, the engineered amphiphilic composition comprises L-α-phosphatidylcholine (PC), Cholesterol, and DSPE-PEG-maleimide.

In embodiments, the multiple CD47 protein are conjugated to an amphiphiles comprising a carboxylic acid, for example, DSPE-PEG-carboxylic acid. Alternatively, the CD47 with a cysteine can be conjugated using thiol-maleimide conjugation chemistry to an amphiphile comprising a maleimide group, for example, DSPE-PEG-maleimide.

One aspect provides a method to treat cancer comprising administering the engineered amphiphilic composition to a subject in need thereof.

Another aspect provides a method to enhance the phagocytic function of macrophages comprising contacting, in vitro or in vivo, said macrophages by contacting a cancer cell with the engineered amphiphilic composition.

One aspect further provides administering to a subject in need thereof, or contacting a cancer cell with, one or more additional anti-cancer agents. In one aspect the one or more additional anti-cancer agents are selected from a small molecule (including, but not limited to, chemotherapeutic agents), antibody, protein, peptide, radiation, surgery, or an immune checkpoint inhibitor. In embodiments, the one or more additional anticancer agents is one or more checkpoint inhibitors (e.g., one or more checkpoint inhibitors disclosed herein). In embodiments, the one or more checkpoint inhibitors is an anti-PDL1 antibody (e.g., atezolizumab, avelumab, and/or durvalumab) and/or an anti-PD1 antibody (e.g., pembrolizumab, nivolumab, and/or cemiplimab). In embodiments, the one or more check-point inhibitors is an inhibitor of cytotoxic T-lymphocyte-associated protein 4 (CTLA4) (e.g., an anti-CTLA4 antibody such as ipilimumab or tremelimumab). In embodiments, the one or more check-point inhibitors is an inhibitor of lymphocyte activation gene-3 (LAG3) (e.g., an anti-LAG3 antibody such as relatlimab). In one aspect, the immune checkpoint inhibitor is an anti-PDL1 antibody. The additional anti-cancer agent(s) may be administered in the same pharmaceutic composition as the engineered amphiphilic composition, or in a separate pharmaceutic composition.

### BRIEF DESCRIPTION OF THE FIGURES

FIGS. 1A, 1B, 1C, and 1D: The multivalent CD47 LNP abrogates tumor growth in anti PDL1 resistant tumors. FIG. 1A: In vivo efficacy studies in immunocompetent B16 melanoma model. FIG. 1B: Graph shows the tumor growth profiles of mice treated with mCD47-PDL1 (mCD47 is multivalent CD47) and other treatments. Data shown are mean±s.e.m. (n=5). FIG. 1C: Graphs quantifying the expression of different effector T-cell markers (CD3⁺ CD8⁺) from a single cell suspension of excised tumors. FIG. 1D: Graphs show quantification of the M1 marker (CD45⁺CD11b⁺CD80) from a single cell suspension of excited tumors. Data shown are mean±s.e.m. (n=3). Statistical significance was determined using one-way ANOVA with Tukey post-test. **p<0.01, ****p<0.0001.

FIG. 2: Treatment with multivalent-CD47 results in significant enhancement in phagocytic ability of macrophages as compared to other treatments. The graph shows quantification of the phagocytic index. i.e., the percentage of phagocytic macrophages in a macrophage-cancer cells co-culture assay. Briefly, macrophages were stained with Cell Trace Far Red and incubated with the cancer cells that were stained with Cell Trace CFSE. The co-culture was treated with the different treatments for 4 h, the cells were then collected, washed twice and flow cytometric analysis was performed. (n=3, Mean±SEM). Statistical analysis is performed with one-way ANOVA followed by Tukey post-test. **p<0.01.

FIG. 3: This figure shows the internalization of FITC dye-tagged engineered multivalent CD47 protein in macrophages at different time points. The nuclei are stained with DAPI.

### DETAILED DESCRIPTION

### Overview of Composition/Methods

Macrophages are cells of the body that exist in either a pro inflammatory M1 phenotype capable of mounting an attack against incoming pathogens or can exist in an anti-inflammatory M2 phenotype that maintains homeostasis in tissues. However, in many solid tumors, the tumor associated macrophages (TAMs) exist in an M2 phenotype, which is pro tumorigenic in nature and supports the growth and metastasis of tumors. This pro tumorigenic phenotype arises due to the cocktail of immunosuppressive cytokines present in the tumor microenvironment. However, it is possible to therapeutically intervene to convert these macrophages from the pro tumorigenic M2 phenotype to the anti-tumorigenic M1 phenotype. In fact, there have been many such strategies to reprogram TAMs that have been studied both pre clinically and clinically. Strategies involving small molecule inhibitors and antibodies have failed due to (i) non targeted drug delivery (ii) unstained inhibition of signaling pathways involving in M2 polarization (iii) off target effect. Moreover, tumor cells use yet another strategy to evade macrophage immunotherapy wherein they express the cell surface protein CD47. The CD47 protein binds to the myeloid cell surface protein SIRPα expressed on macrophages, hence initiating the “eat me not” signaling cascade, which in turn deters macrophages from eating and killing cancer cells through the process of phagocytosis.

In order to offset these challenges and improve macrophage immunotherapy to solid tumors, a two-prong strategy was developed. First, a multivalent recombinant CD47 protein that is multi-functional in nature was engineered. This offers a unique advantage as compared to conventional antibody or protein delivery techniques since the multimodal nature of the system allows for multiple CD47 protein moieties to be decorated on the surface of the engineered amphiphilic composition thereby allowing it to engage with multiple macrophages at the same time to perform a single advanced function of blocking the SIRPα on macrophages from binding to CD47 on the surface of tumor cells.

Provided herein is the activation of the innate immune system (macrophages) in tandem with the adaptive immune system (T cells). To obtain a synergistic anti-tumor response, engineered amphiphilic therapeutic mCD47 were co-administered along with anti-PDL1 antibodies in order to stimulate T cell-cancer cell killing in tandem with enhanced macrophage phagocytosis.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The terms “individual,” “subject,” and “patient,” are used interchangeably herein and refer to any subject for whom diagnosis, treatment, or therapy is desired, including a mammal. Mammals include, but are not limited to, humans, farm animals, sport animals and pets. A “subject” is a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

The terms “inhibit”, “inhibiting”, and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” is an amount sufficient to effect beneficial or desired result, such as preclinical or clinical results. An effective amount can be administered in one or more administrations.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, the term “pharmaceutically-acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (e.g., an amino sequence of a ligand or a ligand binding domain within a protein); in other words the ligand comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term amphiphile refers to a chemical compound possessing both hydrophilic (water-loving, polar) and lipophilic (fat-loving) properties. Such a compound is called amphiphilic or amphipathic. Common amphiphilic substances are soaps, detergents, and lipoproteins. The phospholipid amphiphiles are the major structural component of cell membranes.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

As used herein, an “instructional material” includes a publication, a recording, a diagram, or any other medium of expression which can be used to communicate the usefulness of the of the engineered a in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified invention or be shipped together with a container. Alternatively, the instructional material may be shipped separately from the container with the intention that the instructional material and the invention be used cooperatively by the recipient.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### I. CD47

CD47 (Cluster of Differentiation 47) also known as integrin associated protein (IAP) is a transmembrane protein that in humans is encoded by the CD47 gene. CD47 belongs to the immunoglobulin superfamily and partners with membrane integrins and also binds the ligands thrombospondin-1 (TSP-1) and signal-regulatory protein alpha (SIRPα). CD-47 acts as a “don't eat me” signal to macrophages of the immune system.

CD47 is involved in a range of cellular processes, including apoptosis, proliferation, adhesion, and migration. Furthermore, it plays a role in immune and angiogenic responses. CD47 is expressed in human cells and has been found to be overexpressed in many different tumor cells, human and animal. CD47 interacts with signal-regulatory protein alpha (SIRPα), an inhibitory transmembrane receptor present on myeloid cells. The CD47/SIRPα interaction leads to bidirectional signaling, resulting in different cell-to-cell responses including inhibition of phagocytosis, stimulation of cell-cell fusion, and T-cell activation.

CD47 is a 50 kDa membrane receptor that has an extracellular N-terminal IgV domain, five transmembrane domains, and a short C-terminal intracellular tail. There are four alternatively spliced isoforms of CD47 that differ only in the length of their cytoplasmic tail.

Human CD47 sequences are known in the art. Accession numbers, include, but are not limited to, mRNA: NM_001025079; NM_001025080; NM_001777; NM_198793 and NM_001382306; protein: NP_001768; NP_942088 and NP_001369235. These sequences are incorporated herein by reference. Sequences with 80%, 85%, 90%, 95%, 96%, 97%, 98%, 99% and 100% sequence identity to those sequences are also included herein.

In embodiments the CD47 protein comprises the human CD47 amino acid sequence:

(SEQ ID NO: 1)
MWPLVAALLLGSACCGSAQLLFNKTKSVEFTFCNDTVVIPCFVTN
MEAQNTTEVYVKWKFKGRDIYTFDGALNKSTVPTDFSSAKIEVSQ
LLKGDASLKMDKSDAVSHTGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVFSTAIGLTSFVIAILVIQVIAYILAVVGLSLCIAACIPMHGPL
LISGLSILALAQLLGLVYMKFVASNQKTIQPPRKAVEEPLNAFKE
SKGMMNDE

In embodiments, the CD47 protein comprises an isoform of the above human CD47 protein comprising the amino acid sequence:

(SEQ ID NO: 2)
MWPLVAALLLGSACCGSAQLLFNKTKSVEFTFCNDTVVIPCFVTN
MEAQNTTEVYVKWKFKGRDIYTFDGALNKSTVPTDFSSAKIEVSQ
LLKGDASLKMDKSDAVSHTGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVFSTAIGLTSFVIAILVIQVIAYILAVVGLSLCIAACIPMHGPL
LISGLSILALAQLLGLVYMKFV
(SEQ ID NO: 3)
MWPLVAALLLGSACCGSAQLLFNKTKSVEFTFCNDTVVIPCFVTN
MEAQNTTEVYVKWKFKGRDIYTFDGALNKSTVPTDFSSAKIEVSQ
LLKGDASLKMDKSDAVSHTGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVFSTAIGLTSFVIAILVIQVIAYILAVVGLSLCIAACIPMHGPL
LISGLSILALAQLLGLVYMKFVASNQKTIQPPRNN
(SEQ ID NO: 4)
MWPLVAALLLGSACCGSAQLLFNKTKSVEFTFCNDTVVIPCFVTN
MEAQNTTEVYVKWKFKGRDIYTFDGALNKSTVPTDFSSAKIEVSQ
LLKGDASLKMDKSDAVSHTGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVFSTAIGLTSFVIAILVIQVIAYILAVVGLSLCIAACIPMHGPL
LISGLSILALAQLLGLVYMKFVASNQKTIQPPRKAVEEPLN

In embodiments, the CD47 protein is a human CD47 protein comprising one of the following sequences:

(SEQ ID NO: 5)
QLLFNKTKSVEFTFCNDTVVIPCFVTNMEAQNTTEVYVKWKFKGR
DIYTFDGALNKSTVPTDFSSAKIEVSQLLKGDASLKMDKSDAVSH
TGNYTCEVTELTREGETIIELKYRVVSWFSPNENILIVIFPIFAI
LLFWGQFGIKTLKYRSGGMDEKTIALLVAGLVITVIVIVGAILFV
PGEYSLKNATGLGLIVTSTGILILLHYYVFSTAIGLTSFVIAILV
IQVIAYILAVVGLSLCIAACIPMHGPLLISGLSILALAQLLGLVY
MKFVASNQKTIQPPRKAVEEPLNAFKESKGMMNDE
(SEQ ID NO: 6)
QLLFNKTKSVEFTFCNDTVVIPCFVTNMEAQNTTEVYVKWKFKGR
DIYTFDGALNKSTVPTDFSSAKIEVSQLLKGDASLKMDKSDAVSH
TGNYTCEVTELTREGETIIELKYRVVSWFSPNENILIVIFPIFAI
LLFWGQFGIKTLKYRSGGMDEKTIALLVAGLVITVIVIVGAILFV
PGEYSLKNATGLGLIVTSTGILILLHYYVFSTAIGLTSFVIAILV
IQVIAYILAVVGLSLCIAACIPMHGPLLISGLSILALAQLLGLVY
MKFV
(SEQ ID NO: 7)
QLLFNKTKSVEFTFCNDTVVIPCFVTNMEAQNTTEVYVKWKFKGR
DIYTFDGALNKSTVPTDFSSAKIEVSQLLKGDASLKMDKSDAVSH
TGNYTCEVTELTREGETIIELKYRVVSWFSPNENILIVIFPIFAI
LLFWGQFGIKTLKYRSGGMDEKTIALLVAGLVITVIVIVGAILFV
PGEYSLKNATGLGLIVTSTGILILLHYYVFSTAIGLTSFVIAILV
IQVIAYILAVVGLSLCIAACIPMHGPLLISGLSILALAQLLGLVY
MKFVASNQKTIQPPRNN
(SEQ ID NO: 8)
QLLFNKTKSVEFTFCNDTVVIPCFVTNMEAQNTTEVYVKWKFKGR
DIYTFDGALNKSTVPTDFSSAKIEVSQLLKGDASLKMDKSDAVSH
TGNYTCEVTELTREGETIIELKYRVVSWFSPNENILIVIFPIFAI
LLFWGQFGIKTLKYRSGGMDEKTIALLVAGLVITVIVIVGAILFV
PGEYSLKNATGLGLIVTSTGILILLHYYVFSTAIGLTSFVIAILV
IQVIAYILAVVGLSLCIAACIPMHGPLLISGLSILALAQLLGLVY
MKFVASNQKTIQPPRKAVEEPLN

In embodiments, the CD47 protein comprises the amino acid sequence of SEQ ID NO: 1-8 and sequences with 80%, 85%, 90%, 95%, 96%, 97%, 98%, 99% and 100% sequence identity to SEQ ID NO: 1-8.

In embodiments, the CD47 protein is linked (directly or indirectly) to a protein tag, including, for example, a Myc tag (EQKLISEEDL (SEQ ID NO: 9). In embodiments, the protein tag is linked to the CD47 protein via an amino acid linker. Amino acid linkers can include, for example a GS linker, which is a polypeptide comprising G and S residues. In embodiments, the linker comprises GGGGS. Thus, in one embodiment

### II. Multivalent CD47/Engineered Amphiphilic Composition

Provided herein is a multivalent CD47 particle; a particle with multiple CD47 molecules available for binding. These multiple CD47 proteins are located on the surface of an amphiphile, such as a lipid molecule, to form an engineered amphiphilic composition, and are available for binding (e.g., to SIRPα).

The engineered amphiphilic composition can comprise as the lipid component, one or more of phospholipids, pegylated phospholipids, and cholesterol. The composition may optionally comprise one or more polymers.

The engineered amphiphilic composition can include, but is not limited to, one or more of dioleylphosphateidylethanolamine, phosphatidyl choline (PC), including L-α-phosphatidylcholine, and/or dioleylphosphatidylcholine, 1,2-dioleoyl-sn-glycero-3-phosphocholine, dipalmitoylphosphatidylcholine, 1,2-distearoyl-sn-glycero-3-phosphoethanolamine-N-[carboxy(polyethylene glycol)-2000], 1,2-distearoyl-sn-glycero-3-phosphoethanolamine-N-[carboxy(polyethylene glycol)-2000, NHS ester], 1,2-dioleoyl-sn-glycero-3-phosphoethanolamine-N-[carboxy(polyethylene glycol)-2000], 1,2-Dimyristoyl-sn-glycero-3-phosphocholine, 1,2-dioleoyl-sn-glycero-3-phospho-(1′-rac-glycerol), 1,2-distearoyl-sn-glycero-3-phosphoethanolamine (DSPE), polymers such as poly(lactic-co-glycolic) acid, poly(lactic acid), polyethylene glycol, polyethylenimine, polymethylmethacrylate, polyhydroxyalkanoate, triblock copolymers of poly(ethylene oxide) and/or poly(propylene oxide) and/or cholesterol. The lipids can be pegylated, for example, 1,2-distearoyl-sn-glycero-3-phosphoethanolamine-N-(amino(polyethylene glycol)-2000).

In embodiments, engineered amphiphilic composition can include, one or more of 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-dinonadecanoyl-sn-glycero-3-phosphocholine, 1,2-diheptadecanoyl-sn-glycero-3-phosphocholine, 1,2-dipentadecanoyl-sn-glycero-3-phosphocholine, 1,2-ditridecanoyl-sn-glycero-3-phosphocholine, 1,3-dipalmitoyl-rac-glycero-2-phosphocholine, 1,2-diarachidoyl-sn-glycero-3-phosphocholine, 1-palmitoyl-2-oleoyl-glycero-3-phosphocholine, L-α-phosphatidylcholine (Soy), L-α-phosphatidylcholine (95%) (Egg, Chicken), 1,2-distearoyl-sn-glycero-3-phosphoethanolamine-N-[carboxy(polyethylene glycol)-2000, NHS ester] (sodium salt), 1,2-distearoyl-sn-glycero-3-phosphoethanolamine-N-[maleimide(polyethylene glycol)-2000] (ammonium salt) (DSPE-PEG(2000) Maleimide), 1,2-distearoyl-sn-glycero-3-phosphoethanolamine-N-[succinyl(polyethylene glycol)-2000] (ammonium salt), 1,2-distearoyl-sn-glycero-3-phosphoethanolamine-N-[maleimide(polyethylene glycol)-5000] (ammonium salt) (DSPE-PEG(5000) Maleimide), 1,2-dioleoyl-sn-glycero-3-phosphoethanolamine-N-[carboxy(polyethylene glycol)-2000] (sodium salt) (DOPE-PEG(2000) Carboxylic acid), 1,2-distearoyl-sn-glycero-3-phosphoethanolamine-N-[carboxy(polyethylene glycol)-1000] (sodium salt) (DSPE-PEG(1000) Carboxylic Acid).

Components of the lipid component of the engineered amphiphilic composition may comprise one or more of L-α-phosphatidylcholine (PC), Cholesterol, and 1,2-Distearoyl-sn-Glycero-3-Phosphoethanolamine-N-(Carboxy (Polyethylene Glycol)2000) (DSPE-PEG-Carboxylic Acid).

In embodiments, the lipid component of the engineered amphiphilic therapeutic composition comprises, consists essentially of, or consists of one or more of phospholipids, one or more pegylated phospholipids and cholesterol. The pegylated phospholipid(s), cholesterol, and phospholipid(s) may be present in molar ratios of about 1:1:8, about 1:3:6, or about 1:4:5. In embodiments, the pegylated phospholipid(s) comprise about 5 to about 30 mol % of the lipid component; the cholesterol comprises about 5 to about 40 mol % of the lipid component; and the phospholipid(s) comprise about 40 to about 80 mol % of the lipid component.

In embodiments, the lipid component of the engineered amphiphilic therapeutic composition comprises, consists essentially of, or consists of DSPE-PEG-Carboxylic Acid, Cholesterol and PC. The DSPE-PEG-Carboxylic Acid, Cholesterol and PC may be present in molar ratios of 1:1:8, 1:3:6, or 1:4:5. In embodiments, the DSPE-PEG-Carboxylic Acid comprise about 5 to about 30 mol % of the lipid component, cholesterol comprises about 5 to about 40 mol % of the lipid component; and PC comprises about 40 to about 80 mol % of the lipid component.

In embodiments, the engineered amphiphilic composition comprises, consists of, or consists essentially of a lipid nanoparticle (LNP) that is linked to the multiple CD47 proteins. In embodiments, the multivalent-CD47 LNPs (i.e., LNPs comprising multiple CD47 proteins) do not comprise a payload (e.g., a nucleic acid encoding a transgene or a separate therapeutic protein).

The CD47 protein can be conjugated to a component of the LNP (e.g., an amphiphile or lipid component disclosed herein) using EDC-NHS conjugation chemistry where amine on the protein can be covalently conjugated to the carboxylic acid on the LNPs that available, for example, from the DSPE-PEG-carboxylic acid component. The CD47 with a cysteine can be conjugated on the LNPs using thiol-maleimide conjugation chemistry, the maleimide group will be available, for example, from a DSPE-PEG-maleimide component (in this case, for example, DSPE-PEG-maleimide is used for LNP synthesis instead of DSPE-PEG-Carboxylic acid).

In embodiments, the amphiphilic composition (e.g., LNPs) comprising multiple CD47 proteins are of an average particle size of between about 40 nm and about 300 nm (e.g., about 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm). In some embodiments, the average mean particle size of the engineered amphiphilic multivalent CD47 composition (e.g., multivalent CD47 LNP) is between about 50 nm to about 250 nm, about 50 nm to about 200 nm, about 100 nm to about 300 nm, about 100 nm to about 200 nm, or about 150 nm to about 250 nm. In embodiments, the average mean particle size of the engineered amphiphilic composition is less than about 300 nm, less than about 250 nm, or less than about 200 nm.

In embodiments, the amphiphilic composition is not a liposome (i.e., is not a vesicle are composed of a lipid bilayer that forms in the shape of a hollow sphere, typically encompassing an aqueous phase that comprises a payload). Likewise, in embodiments, the amphiphilic composition is not a viral particle or virus-derived particle (e.g., from an enveloped virus such as lentivirus).

The multivalent CD47 molecule in the context of the engineered amphiphilic composition described herein binds macrophages more efficiently and reduces toxicity. In embodiments, the engineered amphiphilic composition described herein can be used in a method for increasing phagocytosis of cancer cells by macrophages by contacting the cancer cells with an engineered amphiphilic composition described herein.

### III. Cancer Types

Types of cancer that can be treated using the compositions and methods of the invention include, but are not limited to, solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

### IV. Administration

Aspects of this disclosure provides administration of an engineered amphiphilic composition comprising multiple CD47 proteins alone or in combination (either pre-administration, post-administration or simultaneous administration) with other cancer treatments, including but not limited immune checkpoint inhibitors including anti-PDL1 antibodies, and other anti-cancer agents including but not limited to small molecules, antibodies (including, but not limited anti-PD1, anti-CTAL4), proteins, peptides, cell therapies (including, but not limited to, CART T cell therapies), chemotherapy, radiation, surgery, immunotherapy (which are available to an artworker (such as for purchase from several companies) and/or can be readily made by an artworker).

Aspects of this disclosure include administration to the patient in need thereof one or more checkpoint inhibitors (also called immune checkpoint inhibitors). Examples of checkpoint inhibitors include PDL1 inhibitors, PDL2 inhibitors and PD1 inhibitors. Other examples of checkpoint inhibitors include CTLA4 inhibitors and LAG3 inhibitors.

In embodiments, the one or more check point inhibitors is a PDL1 inhibitor including, for example, an antibody that binds to PDL1. In embodiments, the anti-PDL1 antibody is atezolizumab (Tecentriq), avelumab (Bavencio), or durvalumab (Imfinzi).

In embodiments, the one or more check point inhibitors is a PD1 inhibitor including, for example, an antibody that binds PD1. In embodiments, the anti-PD1 antibody is pembrolizumab (Keytruda), nivolumab (Opdivo), cemiplimab (Libtayo) or dostarlimab-gxly (Jemperli).

In embodiments, the one or more checkpoint inhibitors is a LAG3 inhibitor including, for example, an antibody that binds to LAG3 such as relatlimab. In embodiments, the anti-LAG3 antibody is administered in combination with a PD-1 inhibitor, for example Relatlimab may be administered in combination with nivolumab (in a combination known as Opdualag).

A composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic (such as lyophilized bacteria and bacteria thawed from frozen solution).

For intravenous administration, suitable carriers include physiological saline, bacteriostatic water, Cremophor EL™ (BASF; Parsippany, N.J.) or phosphate buffered saline (PBS). It must be stable under the conditions of manufacture and storage and must be preserved against the contaminating action of microorganisms. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Injectable solutions can be prepared by incorporating the active compound (or DNTs) in the required amount in an appropriate solvent with one or a combination of ingredients discussed above. Generally, dispersions are prepared by incorporating the active compound into a vehicle which contains a basic dispersion medium and various other ingredients discussed above. In the case of powders for the preparation of injectable solutions, methods of preparation are vacuum drying and freeze-drying which yields a powder of the active ingredient plus any additional desired ingredient from a previously.

Oral compositions generally include an inert diluent or an edible carrier. For example, they can be enclosed in gelatin capsules. For the purpose of oral therapeutic administration, the active compound/DNT can be incorporated with excipients and used in the form of tablets, troches, or capsules.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the engineered amphiphilic composition is delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bacteria are formulated into ointments, salves, gels, or creams as generally known in the art.

It can be advantageous to formulate compositions in dosage unit form for ease of administration and uniformity of dosage. Dosage unit form as used herein refers to physically discrete units suited as unitary dosages for the subject to be treated; each unit containing a predetermined quantity of active compound/DNT calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

### EXAMPLES

The following examples are provided in order to demonstrate and further illustrate certain embodiments and aspects of the present invention and are not to be construed as limiting the scope thereof.

### Example 1

### Materials and Methods

### Multivalent CD47 Formulation:

Murine CD47 was used for the mouse studies having the following amino acid sequence:

MWPLAAALLLGSCCCGSAQLLFSNVNSIEFTSCNETVVIPCIVRN
VEAQSTEEMFVKWKLNKSYIFIYDGNKNSTTTDQNFTSAKISVSD
LINGIASLKMDKRDAMVGNYTCEVTELSREGKTVIELKNRTVSWF
SPNEKILIVIFPILAILLFWGKFGILTLKYKSSHTNKRIILLLVA
GLVLTVIVVVGAILLIPGEKPVKNASGLGLIVISTGILILLQYNV
FMTAFGMTSFTIAILITQVLGYVLALVGLCLCIMACEPVHGPLLI
SGLGIIALAELLGLVYMKFVASNQRTIQPPRNRGGGGSEQKLISE
EDL
(SEQ ID NO: 9; where the underlined
sequence is a linker and the
double-underlined sequence is a myc tag).

An engineered amphiphilic composition comprising CD47 proteins were synthesized by lipid-film hydration method. 30 mol % DSPE-PEG Carboxylic Acid, 10 mol % Cholesterol and 60 mol % PC were taken and dissolved in 1 ml of (dichloromethane) DCM. The solvent was evaporated using a rotary evaporator to obtain a thin and uniform film. The film was hydrated for 1.5 h in 1 ml PBS at 60° C. Following this, 1.5 molar equivalents of EDC and NHS were each added and incubated at room temperature for 2 hours. To this, 20 μg of CD47 protein was added to synthesize CD47 LNPs These samples were incubated at 4° C. for 12 h after which they were extruded using 0.4 μm and 0.2 μm Polycarbonate membrane at 60° C. to obtain sub-200 nm particles.

In order to remove free molecular subunits and non-conjugated protein, the samples were passed through a Sephadex G-25 column. The mean particle size and zeta potential was measured using Malvern Zetasizer ZSP. The physical stability of particles was evaluated by measuring the changes in mean size and zeta potential in 40° C. storage conditions.

### Animal Trial:

1×10⁶ B16/F10 melanoma cells were implanted subcutaneously into right flanks of 4-6 weeks old C57BL/6 mice weighing 20 g (Charles River Laboratories). When the tumor size reached ˜75 mm³, treatment was started and was considered as day 0. The drug therapy consisted of administration of CD47 LNP, anti-PDL1 antibody (BioXcell), CD47 LNP+anti-PDL1 antibody, Free CD47 protein and an untreated control every alternate day for a total 3 dosages. All doses were equivalent to 20 μg/mouse of CD47 protein and 10 mg/kg of PDL1. The body weights and tumor volumes of mice were monitored every alternate day until the end of study. Tumor diameters were measured using a Vernier caliper and tumor volumes were calculated using volume of an ellipsoid (L×B2/2), L being the longest diameter of the tumor and B being the shortest). The mice were sacrificed when the tumor size reached 2000 mm³. All the animal procedures were performed in accordance with the institutional guidelines for using and careful handling of laboratory animals of the University of Massachusetts, Amherst, MA, USA and all the procedures were approved by the Animal Ethics Committee of the University of Massachusetts Amherst.

### Results/Discussion

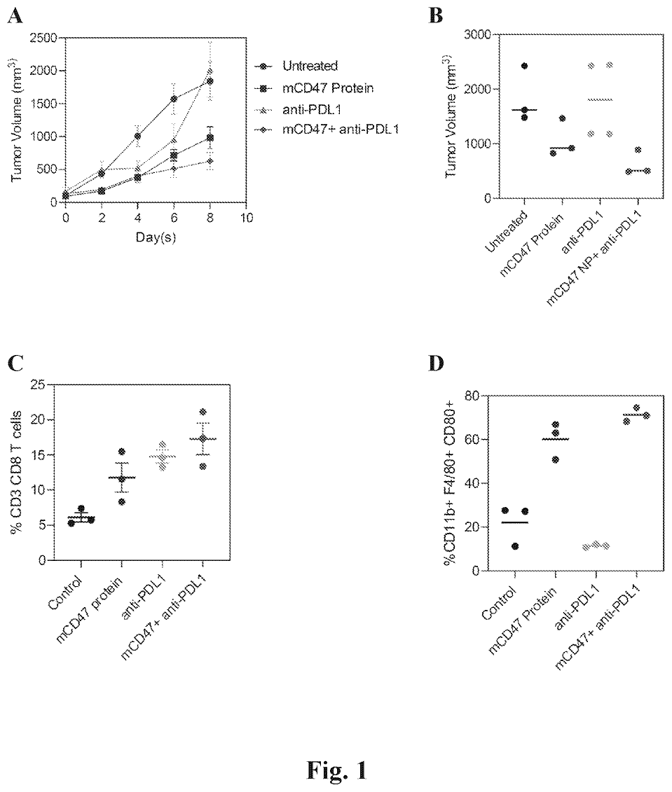

Multivalent CD47 reduced tumor volume in vivo, as did anti-PDL antibody (FIGS. 1A, 1B). The combination of CD47 and anti-PDL1 antibody resulted in a synergistic decrease in tumor volume (FIGS. 1A, 1B). Further, CD3 and CD9 T-cell markers were increased when exposed to multivalent CD47 and anti-PDL1 antibody (FIG. 1C), with a synergistic increase upon exposure to a combination of CD47 and anti-PDL1 antibody (FIG. 1C). While there was a decrease in the M1 marker (CD11b⁺F4/80⁺CD80⁺) for anti-PDL1 antibody (FIG. 1D), there was an increase with multivalent CD47 and a synergistic increase upon exposure to a combination of CD47 and anti-PDL1 antibody (FIG. 1D).

### Example 2

### Materials and Methods

The multivalent CD47 LNPs were synthesized by lipid-film hydration method as described above.

### In-Vitro Assay Evaluating the Phagocytic Index of Macrophages Treated With Multivalent-CD47:

B16/F10 melanoma cells were labelled with CSFE as per the manufacturers protocol (CSFE cell trace labeling kit). The labeled cells can be detected in the green-fluorescent channel. Upon labeling these cells, 2.5×10⁴ B16/F10 cells were seeded per well in an ultra-low attachment non-adherent 96 well plate and cocultured with 5×10⁴ RAW 264.7 macrophages that were stained with Cell Trace Far Red at a ratio of 1:2. The resulting macrophage, cancer cell co-culture, was then incubated for 4 h with different treatments in serum-free media. The cells were collected and analyzed by an ACEA Novocyte flow cytometer. Phagocytosis index was determined by the counting of CSFE positive cells within far red stained macrophage cell gate.

### In Vitro Assay Evaluating the Internalization of Dye-Tagged Multivalent CD47:

RAW264.7 cells were seeded (80×103) onto an 8-well chamber slide. The FITC-tagged multivalent CD47 LNP and the FITC-NP were added at 10 μM dye concentration and incubated for 4 hours. Following this, the wells were washed with PBS and were fixed using 4% paraformaldehyde, stained with DAPI followed by mounting with Invitrogen Glass Antifade reagent. Images were taken using a Nikon A1R-SIMe confocal microscope at 60× and were analyzed using NIS Elements 4.6.

### Results/Discussion

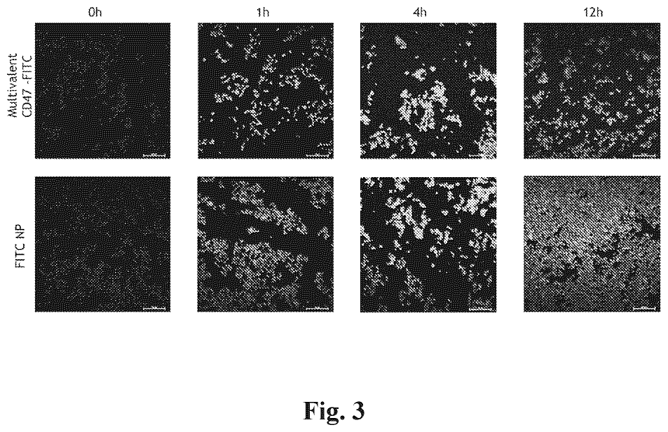

The functional aspect of multivalent CD47 is to enhance the phagocytosis of cancer cells by macrophages. Phagocytosis is a proximity-based phenomenon where macrophages physically engulf cancer cells by initiating a phagocytic synapse. We hypothesized that the engagement of a macrophage and a cancer cell by blocking the “don't eat me” signaling pathway might lead to an enhanced phagocytic index of macrophages. To validate this hypothesis, the binding efficacy of multivalent CD47 to macrophages was evaluated. Multivalent CD47 was synthesized by conjugating equimolar concentrations of FITC dye-tagged CD47 on LNPs, while a control LNP was synthesized by encapsulating FITC dye in the lipid nanoparticle. RAW 264.7 macrophages were treated with either Multivalent CD47 or control LNP for 4-12 h, and the binding and internalization were analyzed using the confocal microscope. An increased binding efficacy was observed by Multivalent CD47 on the surface of macrophages at earlier time points (1 h) when compared to control LNPs (FIG. 3), demonstrating that the multivalent CD47 can efficiently bind to the SIRPα on macrophages early on and results in internalization at a later time points (12 h). The attachment of Multivalent CD47 on the cellular surface proves that conjugated CD47 are functionally active.

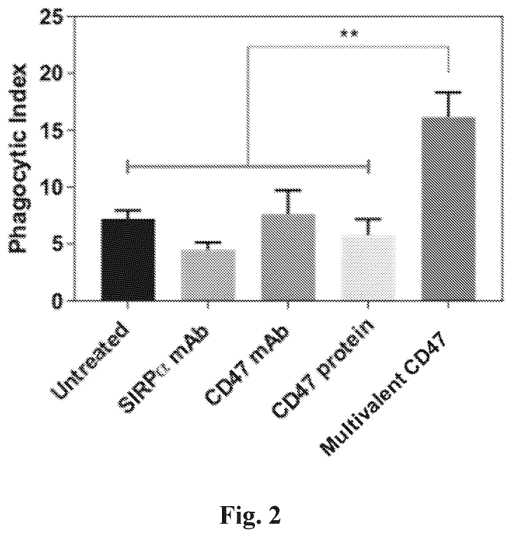

In addition, the effect of treatment with multivalent CD47 on the phagocytosis of cancer cells by macrophages was evaluated in a coculture assay. RAW 264.7 macrophages were added to CFSE-stained B16/F10 melanoma cells and cocultured for 4 h while being subjected to treatment by either multivalent CD47, CD47 mAb, SIRPα mAb or CD47 protein. It was observed that the macrophages treated with multivalent CD47 showed significantly higher phagocytosis of cancer cells than other treatment groups (FIG. 2). Interestingly, macrophages treated with CD47 protein, CD47 mAb and SIRPα antibodies showed a minimal phagocytosis level, comparable to untreated control and significantly lower than multivalent CD47. This observation further validates that efficiently blocking CD47-SIRPα interactions using the engineered multivalent CD47 amphiphilic compositions described herein can further enhance phagocytosis.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. An engineered amphiphilic composition, wherein the engineered amphiphilic composition comprises multiple CD47 proteins (mCD47/multivalent) and at least one amphiphile, wherein the engineered amphiphilic composition binds SIRP-alpha protein.

2. The engineered amphiphilic composition of claim 1, wherein the amphiphile comprises one or more of 1,2-Distearoyl-sn-glycero-3-phosphoethanolamine (DSPE), cholesterol, sitosterol, phosphatidylcholine (PC) or other lipids that are conjugable.

3. The engineered amphiphilic composition of claim 1, wherein the amphiphile is pegylated.

4. The engineered amphiphilic composition of claim 1, further comprising a carrier and/or one or more additional anti-cancer agents.

5. A method to treat cancer comprising administering the engineered amphiphilic composition of claim 1 to a subject in need thereof.

6. A method to enhance the phagocytic function of macrophages comprising contacting, in vitro or in vivo, said macrophages with the engineered amphiphilic composition of claim 1.

7. The method of claim 5, further comprising administering or contacting with one or more additional anti-cancer agents.

8. The method of claim 7, wherein the one or more additional anti-cancer agents are selected from a small molecule (including, but not limited to, chemotherapeutic agents), antibody, protein, peptide, radiation, surgery, or an immune checkpoint inhibitor.

9. The method of claim 8, wherein the immune checkpoint inhibitor is an anti-PDL1 antibody.
